(19) United States
(12) Patent Application Publication
(10) Pub. No.: US 2015/0198602 A1
(43) Pub. Date: Jul. 16, 2015

(54) THERANOSTIC METHOD BASED ON THE DETECTION OF HER2-HER2 DIMERS

(71) Applicants: CISBIO BIOASSAYS, Codolet (FR); INSTITUTE GUSTAVE ROUSSY, Villejuif Cedex (FR)

(72) Inventors: Fabrice Andre, Sceaux (FR); Herve Bazin, Villeneuve Les Avignon (FR); Patrick Garnero, Gaujac (FR); Gerard Mathis, Bagnols Sur Ceze (FR)

(21) Appl. No.: 14/415,490

(22) PCT Filed: Jul. 16, 2013
(86) PCT No.: PCT/FR2013/051700

§ 371 Date: Jan. 16, 2015

(30) Foreign Application Priority Data
Jul. 17, 2012 (FR) 1256902

(51) Int. Cl. G01N 33/574

(52) U.S. Cl. CPC G01N 33/57492; G01N 2333/71

(57) ABSTRACT

The present invention relates to an ex vivo method for determining the susceptibility of a patient suffering from cancer to respond to a therapeutic treatment based on the administration of an antibody specific for the HER2 protein, this method comprising the quantification of HER2-HER2 dimers in a tissue sample from said patient using the FRET technique.

The invention also relates to a kit of reagents for implementing this method.

The invention relates to a theranostic method based on the detection, by means of a TR-FRET approach, i.e. by means of the real-time measurement of the fluorescence emitted when two compatible fluorescent molecules are in proximity to one another, of homodimers consisting of two HER2 proteins (hereinafter “Her2-Her2”). This method aims to determine whether patients suffering from cancer are susceptible to respond to a therapeutic treatment based on the administration of an antibody specific for the HER2 protein, of trastuzumab type. The invention relates in particular to the implementation of this method with tissue samples from patients for whom the result of the determination of HER2 expression by immunohistochemistry had proved to be negative (Herceptest™-negative patients).

### STATE OF THE ART

The HER2 receptor (an acronym of “human epidermial growth factor receptor 2”) is a transmembrane glycoprotein of 185 kDa which belongs to the HER receptor tyrosine kinase family. In 1987, Slamon et al. discovered that HER2 gene amplification affects approximately one third of patients suffering from breast cancers and correlates with a poor vital prognosis (Slamon D J, Clark G M, Wong S G, Levin W J, Ullrich A, et al. (1987) Science 235: 177-182). Subsequent in vitro and in vivo studies have made it possible to characterize the biological consequences of this molecular abnormality, and have shown that HER2 is an oncogen with promotes tumor growth, angiogenesis and the development of metastases. The hypothesis that the inhibition of HER2 might be an effective therapeutic strategy for the treatment of tumors overexpressing this protein has therefore been put forward. This has led to the production of trastuzumab, a humanized recombinant anti-HER2 monoclonal antibody, which has demonstrated its clinical efficacy in patients suffering from breast tumors overexpressing the HER2 protein, whether in a metastatic or adjuvant context.

In parallel, theranostic tests have been produced for evaluating the expression of the HER2 protein in order to identify patients who might benefit from treatment with the anti-HER2 therapeutic antibody trastuzumab. In routine clinical practice, the quantification of HER2 expression has become obligatory for selecting patients eligible for this treatment. The HER2 test is usually carried out by staining using immunohistochemistry (IHC, for example with the test sold by the company Dako under the name Herceptest™) which makes it possible to detect an overexpression of the HER2 protein, or by FISH (an acronym of “fluorescent in situ hybridization”), for detecting an amplification of the HER2 gene. The IHC approach is semi-quantitative and makes it possible to classify patients on a scale from IHC 0 (negative) to IHC 3+ (strongly positive). This classification is based on the percentage of malignant stained cells and the degree of staining of the membrane of these cells. The Herceptest™ is considered to be negative if the patients are classified 0, 1+ or 2+/FISH−, and only patients for whom the result is 3+ or 2+/FISH+ are eligible for treatment with trastuzumab.

It is known that the activity of the HER2 protein involves its dimerization, in particular involves the formation of HER2-HER2 homodimers. Authors have thus focused on the presence of HER2-HER2 dimers in Herceptest™-positive patients treated with trastuzumab. It has, for example, been shown in retrospective studies that patients having high levels of Her2-Her2 dimers exhibited a longer overall survival and a lengthier progression of the disease than the other patients when they were treated with trastuzumab (Gosh Cancer Res 2011, Desmet et al. Diagn Mol patho 2009).

Patent application WO 2009/086197 relates in particular to a method aimed at determining whether patients are eligible for treatment with an anti-HER2 antibody or else for predicting the efficacy of such treatment, this method being based on the quantification of HER2 or of HER2-HER2 homodimers. This method is solely intended to be implemented on patients for whom the expression tests by IHC or the amplification tests by FISH were positive.

The conventional approaches for detecting HER2 dimers are based on chemical immunoprecipitation and crosslinking techniques which have a low throughput and are not suitable for use in a clinical context, since they require a large amount of proteins.

Other methods have been proposed, in particular by the company Monogram Biosciences which has developed the VeraTag™ test (Desmedt C et al. 2009 Diagn Mol Pathol 18: 22-29, WO 2009/086197) for quantifying HER proteins and protein dimers in FFPE tissue samples. This technique, also known as “eTag”, is based on the use of two antibodies specific for the molecule(s) to be detected, one being coupled, via an oxidation-sensitive bond, to a fluorescent electrophoretic marker, and the other being conjugated to an entity capable of generating singlet oxygen. When the two antibodies are in proximity to one another, the singlet oxygen generated by illumination of the sample will lead to the release of the electrophoretic marker. The markers released are then separated by electrophoresis. This relatively laborious technique requires that the clinical analysis laboratories send their samples to the company Monogram for analysis.

The assay sold under the name Duolink™ enables the detection of Her2-Her2 dimers using antibodies coupled to oligonucleotides. When the antibodies are separated by less than 40 nm, a complex succession of hybridization and amplification steps results in the visualization of the dimers by observation by in situ generated fluorescence microscopy.

Gaborit et al. (J Biol Chem. 2011 Apr 1; 286(13):11337-45) have in particular described the detection of Her2-Her2 dimers at the surface of intact cells using a TR-FRET approach, i.e. by real-time detection of the fluorescence emitted when two compatible fluorescent molecules, each bonded to an anti-HER2 antibody, are in proximity to one another. This method is not suitable for use on tissue, in a clinical context.

Despite the progress made in the therapeutic treatment of patients suffering from breast cancer, those who have been judged non-eligible for treatment of trastuzumab type (for example patients for whom the Herceptest™ or FISH tests were negative) and whose hormone chemotherapy treatment has failed are unfortunately at a therapeutic impasse. The invention makes it possible to offer these patients a new treatment alternative.

### DESCRIPTION OF THE FIGURES

FIG. 1 represents the overall survival probability as a function of time for patients suffering from a breast tumor who were Herceptest™-negative and ER-positive.

FIG. 2 represents the disease-free survival probability as a function of time for patients suffering from a breast tumor who were Herceptest™-negative and ER-positive.

### DESCRIPTION OF THE INVENTION

The inventors have discovered that a TR-FRET approach for detecting and quantifying HER2-HER2 homodimers in tissue samples from patients for whom the result of the determination of HER2 expression by immunohistochemistry had proved to be negative (“IHC negative”, and in particular Herceptest™-negative, patients) makes it possible to reveal new patients eligible for treatment with a Her2-specific antibody, of trastuzumab type.

In a first aspect, the invention relates to an (ex vivo) method for determining the susceptibility of a patient suffering from cancer to respond to a therapeutic treatment based on the administration of an antibody specific for the HER2 protein, this method comprising the quantification of HER2-HER2 dimers in a tissue sample from said patient,
said quantification being carried out by time-resolved measurement of the fluorescence emitted by a pair of FRET partners brought into contact with said sample, the first member of this pair being directly or indirectly bonded to a first ligand capable of binding to a domain of the HER2 protein, the second member of this pair being directly or indirectly bonded to a second ligand capable of binding to the same domain of the HER2 protein as the first ligand, and
the patient being a patient for whom the results of the determination of HER2 expression by immunohistochemistry had proved to be negative, in particular the patient being a Herceptest™-negative patient.

The term “pair of FRET partners” is intended to mean a pair consisting of an energy donor fluorescent compound (hereinafter “donor fluorescent compound”) and an energy acceptor compound (hereinafter “acceptor compound”); when they are in proximity to one another and when they are excited at the excitation wavelength of the donor fluorescent compound, these compounds emit a FRET (acronym of the expression “Förster Resonance Energy Transfer”) signal.

The term “FRET signal” is intended to mean any measurable signal representative of a FRET between a donor fluorescent compound and an acceptor compound. A FRET signal can therefore be a variation in the intensity or in the lifetime of luminescence of the donor fluorescent compound or of the acceptor compound when the latter is fluorescent. When the FRET signal is measured in real time (which is generally the case when rare earth chelates or cryptates are used), the term TR-FRET (acronym of “time-resolved” FRET) is used.

The term “tissue sample” is preferably intended to mean a solid tissue sample taken from a patient, and preferably a tumor tissue extract. This tissue sample is preferably used in the form of sections of 10 to 50 μm thick. These sections are prepared according to the conventional techniques known to those skilled in the art. They can consist in particular of the fixing of the sample by means of a treatment with formaldehyde, and the embedding of said sample in paraffin, in particular in the form of blocks which can be subsequently cut on a microtome (preferably with a thickness of approximately 10 to 30 μm). The treatment of these sections with xylene in order to remove the paraffin, and the rinsing of said sections with ethanol and then with water are also techniques known to those skilled in the art, as is the regeneration of the epitopes using the HIER (acronym for “heat induced epitope recovery”) technique.

Alternatively, the method according to the invention can also be implemented on cryosections, preferably from 20 to 50 μm thick, also prepared according to conventional techniques.

The term “quantification” is intended to mean the obtaining of a signal, the value of which depends on the amount of HER2-HER2 dimers present in the sample. It is interesting to note that, according to the invention, it is possible but not necessary to compare the value representing the amount of HER2 dimers with a threshold value, that observed in reference patients, and above which the patients tested are eligible for treatment with an anti-HER2 antibody. Nevertheless and quite surprisingly, it has been determined that the sole presence of HER2 dimers correlates with a negative prognosis and therefore makes these patients eligible for treatment with an anti-HER2 antibody. In any event, this makes the method according to the invention very practical for hospital personnel, since it allows them to very easily reach a conclusion as to the eligibility of patients for this type of treatment, either because HER2 dimers are detected, or because the amount of dimers detected is above a threshold value determined in reference patients.

The method according to the invention is particularly advantageous when it is implemented with a tissue sample from a patient having been the subject of hormone chemotherapy (patients termed “ER+”, positive for expression of the estrogen receptor), in particular when this approach has not led to any improvement in the patient's condition, since it makes it possible to offer said patient a new therapeutic alternative, even if the result of the determination of HER2 expression by immunohistochemistry (Herceptest™) had proved to be negative, i.e. the samples from these patients had been classified with a score of 0, 1+ or 2+/FISH− in this test.

In one preferred implementation, the first and the second ligand are antibodies or aptamers, and are specific for the same epitope located in the extracellular or intracellular part of the HER2 protein, and these ligands are, in a particularly advantageous implementation, identical. The antibodies or aptamers of which the epitope is located in the extracellular part of HER2 are preferred. The term “antibody” should here be taken in the broad sense and comprises any protein of the immunoglobulin family or else comprising a domain of an immunoglobulin, and also a site for specific binding to the protein of interest. The antibodies may therefore be Fab fragments, Fab′ fragments, single-chain antibodies, or variable domains of immunoglobulin heavy or light chains. Those skilled in the art are able to produce antibodies specific for the HER2 protein using conventional techniques. Anti-HER2 antibodies are also commercially available.

Moreover, it is desirable for the final concentrations of first and second ligands in the incubation medium to be, optimally, greater than or equal to 10 nM, preferably included in the range from 10 to 150 nM, more preferably in the range from 20 to 80 nM, and particularly preferably in the range from 30 to 60 nM. The term “final concentration” is intended to mean the concentration of these compounds in the incubation medium once all the reagents have been introduced into this medium. These concentration ranges are notably higher than the concentrations normally used in assays of TR-FRET type, in which the final concentrations of fluorescent ligands are of the order of one nanomolar, i.e. less than 10 nM.

The quantification of the HER2-HER2 dimers preferentially comprises the following steps:
(i) bringing the tissue sample into contact with the pair of FRET partners bonded to the HER2 ligands;
(ii) washing the tissue sample;
(iii) measuring the FRET signal emitted by the measuring medium.

It may be advantageous to standardize the FRET signal with respect to the amount of biological material present in the measuring medium. The method according to the invention thus comprises, in a particular implementation, the incubation of the tissue sample with a labeling agent which emits a signal proportional to the amount of biological material present in the sample. Preferably, this labeling agent is a fluorescent-DNA labeling agent (for example Hoechst 33342), added to the sample prior to the washing step, and the FRET signal will be standardized with respect to the signal corresponding to the fluorescence of this labeling agent.

The standardization can also be carried out using other types of labeling agents, in particular:
fluorescent compounds which are markers for mitochondria, such as Mitotracker Orange, Mitotracker Green, or else rhodamine 123. These compounds are commercially available and the protocols for labeling cells with these compounds are also known. They can be used according to the invention with a protocol similar to the case where the labeling agent is an intercalating agent;
fluorescent compounds, such as dansyl chloride or NBD (nitrobenzoxadiazole): these compounds, which are also commercially available, bind to amine functions, in particular of proteins. Y. Uratani et al. describe a protocol for labeling cells with dansyl chloride (Journal of Bacteriology 1982 p. 523-528). Once the cells have been labeled, the invention can be implemented with a protocol similar to the case where the labeling agent is an intercalating agent;
fluorescent compounds which accumulate in lipid membranes, such as Filipin: this compound is commercially available and its use for labeling cells is known. It can be used according to the invention with a protocol similar to that of the case where the labeling agent is an intercalating agent.

Finally, it is advantageous to include, in the method for quantifying HER2 dimers, a step aimed at homogenizing this sample in the form of a cell lysate, before or after the introduction of the fluorescent compounds into the measuring medium. This step is preferably carried out after the introduction of the fluorescent compounds into the incubation medium (first ligand, second ligand and optionally labeling agent), and before the measurement of the FRET signal. Such a treatment may be mechanical, and may be chosen from: the application of ultrasound (sonication), freezing/thawing cycles, the use of mechanical grinders, optionally together with the use of a hypotonic lysis buffer or a lysis buffer containing detergents, such as the RIPA buffer.

Preferably and in order to ensure the sensitivity of the method of quantification by TR-FRET, the donor compound is a rare earth chelate or cryptate, in particular a europium or terbium chelate or cryptate, and the acceptor compound is chosen from allophycocyanins, rhodamines, cyanines, squaraines, coumarins, proflavins, acridines, fluoresceins, boron-dipyrromethene derivatives, fluorophores known under the name “Atto”, fluorophores known under the name “DY”, compounds known under the name “Alexa” and nitrobenzoxadiazole.

In a second aspect, the invention relates to a kit of reagents for implementing the method described above, said kit comprising a first ligand and a second ligand, each of these ligands being capable of binding specifically to the same domain of the HER2 protein, and these ligands being respectively directly labeled or suitable for indirect labeling with a donor compound and an acceptor compound, said donor and acceptor compounds forming a pair of FRET partners. Preferably, at least one of these ligands is covalently (directly) labeled with one of the FRET partners, and preferably each of the two ligands is covalently labeled with one of the FRET partners.

The donor compound is preferably a rare earth chelate or cryptate, in particular a europium or terbium chelate or cryptate, and the acceptor compound is preferably chosen from allophycocyanins, rhodamines, cyanines, squaraines, coumarins, proflavins, acridines, fluoresceins, boron-dipyrromethene derivatives, fluorophores known under the name “Atto”, fluorophores known under the name “DY”, compounds known under the name “Alexa” and nitrobenzoxadiazole.

Labeling of the Ligands or Antibodies with Energy Donor or Acceptor Compounds

The ligands can be labeled with the fluorophores directly (covalently) or indirectly. The direct labeling is preferred.

The direct labeling of a ligand or of an antibody with a fluorescent donor or acceptor compound is carried out using the conventional techniques of conjugation which call for the use of reactive groups. The fluorescent donor or acceptor compounds are generally sold in “functionalized” form, i.e. they bear a reactive group capable of reacting with a functional group present on the compound to be labeled, in this case the ligand.

Typically, the reactive group present on the donor or acceptor fluorescent compound is an electrophilic or nucleophilic group which can form a covalent bond when it is placed in the presence of a suitable nucleophilic or electrophilic group, respectively. By way of examples, the pairs of electrophilic/nucleophilic groups and the type of covalent bond formed when they are brought together are listed below:

| Electrophilic group | Nucleophilic group | Bond type |
|---|---|---|
| acrylamide | thiol | thioether |
| acyl halide | amine/aniline | carboxamide |
| aldehyde | amine/aniline | imine |
| aldehyde or ketone | hydrazine | hydrazone |
| aldehyde or ketone | hydroxylamine | oxime |
| alkyl sulfonate | thiol | thioether |
| anhydride | amine/aniline | carboxamide |
| aryl halide | thiol | thiophenol |
| aryl halide | amine | arylamine |
| aziridine | thiol | thioether |
| carbodiimide | carboxylic acid | N-acyl urea or |
| | | anhydride |
| activated ester* | amine/aniline | carboxamide |
| haloacetamide | thiol | thioether |
| halotriazine | amine/aniline | aminotriazine |
| imido ester | amine/aniline | amidine |
| isocyanate | amine/aniline | urea |
| isothiocyanate | amine/aniline | thiourea |
| maleimide | thiol | thioether |
| sulfonate ester | amine/aniline | alkylamine |
| sulfonyl halide | amine/aniline | sulfonamide |

*the term “activated ester” is intended to mean groups of formula COY, where Y is: a leaving group, chosen from succinimidyloxy (—OC₄H₄NO₂) and sulfo-succinimidyloxy (—OC₄H₃NO₂—SO₃H) groups; an aryloxy group which is unsubstituted or substituted with at least one electrophilic substituent, such as nitro, fluoro, chloro, cyano or trifluoromethyl groups, thus forming an activated aryl ester; a carboxylic acid activated with a carbodiimide group, forming an anhydride −OCORa or —OCNRaNHRb, in which Ra and Rb are identical or different and are chosen from C₁-C₆ alkyl, C₁-C₆ perfluoroalkyl, C₁-C₆ alkoxy, and cyclohexyl groups; 3-dimethylaminopropyl or N-morpholinoethyl.

The commercially available donor and acceptor fluorescent compounds generally comprise a maleimide function or an activated ester, most commonly activated with an NHS (N-hydroxysuccinimidyl) group, which react with the thiol and amine groups respectively and can therefore be used for labeling antibodies. The antibodies labeled are characterized by the final molar ratio (FMR) which represents the average number of molecules of label grafted to the ligand.

When the ligand is protein in nature, it may be advantageous to use one of the functional groups naturally present in proteins: the terminal amino group, the terminal carboxylate group, the carboxylate groups of aspartic acid and glutamic acid, the amine groups of lysines, the guanidine groups of arginines, the thiol groups of cysteines, the phenol groups of tyrosines, the indol rings of tryptophans, the thioether groups of methionines, and the imidazole groups of histidines.

If the ligand does not comprise a functional group in the natural state, such groups can be introduced. Methods for introducing functional groups are in particular described in C. Kessler, Nonisotopic probing, Blotting and Sequencing, 2nd edition, L. J. Kricka (1995), Ed. Academic press Ltd., London, p. 66-72.

Another approach for labeling a ligand with a fluorescent compound consists in introducing a reactive group into the ligand, for example an NHS group or a maleimide group, and placing it in the presence of a fluorophore bearing a functional group that will react with the reactive group so as to form a covalent bond.

It is important to verify that the labeled ligand retains sufficient affinity for its receptor; this can be simply controlled with conventional binding experiments, making it possible to calculate the affinity constant of the labeled ligand for the receptor.

The ligand may also be labeled with a fluorescent compound indirectly, for example by introducing into the incubation medium a “secondary” antibody, itself covalently bonded to a fluorescent compound, this antibody specifically recognizing the ligand or else a hapten present on this ligand (such as a dinitrophenyl group, a digoxigenin group, fluorescein, or FLAG, c-myc or 6-HIS tags). When the ligand is an antibody, the secondary antibody may be an anti-species antibody.

Another very conventional means of indirect labeling consists in attaching biotin to the ligand to be labeled, and then incubating this biotinylated ligand in the presence of streptavidin labeled with a fluorophore. The labeling of a ligand with biotin is part of the general knowledge of those skilled in the art, and the company Cisbio Bioassays sells, for example, streptavidin labeled with the fluorophore of which the trade name is “d2” (ref 610SADLA).

Pairs of FRET Partners

The pairs of FRET partners are preferably made up of an energy donor fluorescent compound and an energy acceptor fluorescent compound.

FRET is defined as a transfer of nonradiative energy resulting from a dipole-dipole interaction between an energy donor and an energy acceptor. This physical phenomenon requires energy compatibility between these molecules. This means that the emission spectrum of the donor must at least partially overlap the absorption spectrum of the acceptor. In accordance with Förster's theory, FRET is a process which depends on the distance separating the two donor and acceptor molecules: when these molecules are in proximity to one another, a FRET signal will be emitted.

The selection of the donor / acceptor fluorophore pair for obtaining a FRET signal is within the scope of those skilled in the art. Donor-acceptor pairs usable for studying FRET phenomena are in particular described in the book by Joseph R. Lakowicz (Principles of fluorescence spectroscopy, 2nd edition, Kluwer academic/plenum publishers, NY (1999)), to which those skilled in the art may refer.

Energy donor fluorescent compounds which have a long lifetime (>0.1 ms, preferably between 0.5 and 6 ms), in particular rare earth chelates or cryptates, are advantageous since they make it possible to carry out time-resolved measurements, i.e. to measure TR-FRET signals while dispensing with the phenomenon of auto-fluorescence emitted by the measuring medium. They are for this reason and generally preferred for implementing the method according to the invention.

Dysprosium (Dy3+), samarium (Sm3+), neodymium (Nd3+), ytterbium (Yb3+) or else erbium (Er3+) complexes are rare earth complexes which are also suitable for the purposes of the invention, but europium (Eu3+) and terbium (Tb3+) chelates and cryptates are particularly preferred.

A very large number of rare earth complexes have been described and several are currently sold by the company PerkinElmer, Invitrogen and Cisbio Bioassays.

Examples of rare earth chelates or cryptates which are suitable for the purposes of the invention are:
Lanthanide cryptates, comprising one or more pyridine units. Such rare earth cryptates are described, for example, in patents EP 0 180 492, EP 0 321 353 and EP 0 601 113 and in international application WO 01/96877. Terbium cryptates (Tb3+) and europium cryptates (Eu3+) are particularly suitable for the purposes of the present invention. Lanthanide cryptates are sold by the company Cisbio Bioassays. By way of nonlimiting example, mention may be made of the europium cryptates having the formulae below (which can be coupled to the compound to be labeled by a reactive group, in this case, for example, an NH₂ group):

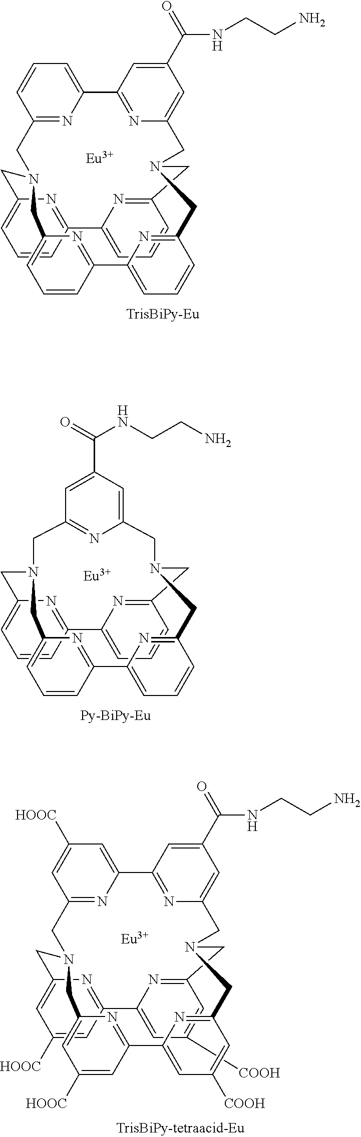

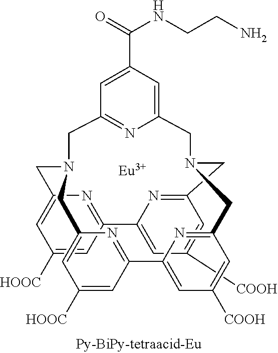

The lanthanide chelates described in particular in patents U.S. Pat. No. 4,761,481; U.S. Pat. No. 5032,677, U.S. Pat. No. 5,055,578, U.S. Pat. No. 5,106,957, U.S. Pat. No. 5,116,989; U.S. Pat. No. 4,761,481; U.S. Pat. No. 4,801,722; U.S. Pat. No. 4,794,191, U.S. Pat. No. 4,637,988, U.S. Pat. No. 4,670,572, U.S. Pat. No. 4,837,169, U.S. Pat. No. 4,859,777. Patents EP 0 403 593, U.S. Pat. No. 5,324,825, U.S. Pat. No. 5,202,423 and U.S. Pat. No. 5,316,909 describe chelates composed of a nonadentate ligand such as terpyridine. Lanthanide cheiates are sold by the company PerkinElmer.
Lanthanide complexes consisting of a chelating agent, such as tetraazacyclododecane, substituted with a chromophore comprising aromatic rings; such as those described by R. Poole et al., in Biomol, Chem, 2005, 3, 1013-1024 “Synthesis and characterisation of highly emissive and kinetically stable lanthanide complexes suitable for usage in cellulo”, can also be used. The complexes described in application WO 2009/10580 can also be used.
The lanthanide cryptates described in patents EP 1 154 991 and EP 1 154 990 are also usable.
The terbium cryptate having the formula below (which can be coupled to a compound to be labeled via a reactive group, in this case, for example, an NH₂ group):

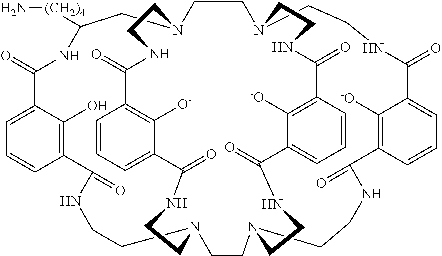

and the synthesis of which is described in the international application WO 2008/063721 (compound 6a, page 89).
The terbium cryptate Lumi4-Tb from the company Lumiphore, sold by Cisbio Bioassays.
The quantum dye from the company Research Organics, having the formula below (which can be coupled to the compound to be labeled via a reactive group, in this case NCS):

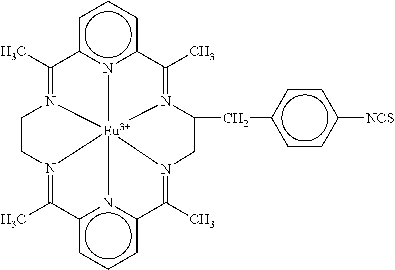

Ruthenium chelates, in particular the complexes consisting of a ruthenium ion and of several bipyridines, such as ruthenium(II) tris(2,2′-bipyridine).
The terbium chelate DTPA-cs124 Tb, sold by the company Life technologies, having the formula below (which can be coupled to the compound to be labeled via a reactive group R) and the synthesis of which is described in U.S. Pat. No. 5,622,821:

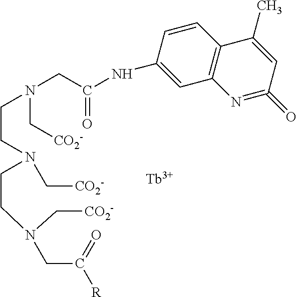

The terbium chelate having the formula below and described by Latva et al. (Journal of Luminescence 1997, 75: 149-169):

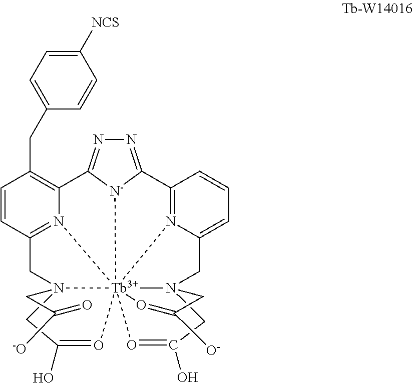

Particularly advantageously, the donor fluorescent compound is chosen from: a europium cryptate; a europium chelate; a terbium chelate; a terbium cryptate; a ruthenium chelate; and a quantum dye; the europium and terbium chelates and cryptates being particularly preferred.

Dysprosium (Dy3+), samarium (Sm3+), neodymium (Nd3+), ytterbium (Yb3+) or else erbium (Er3+) complexes are also rare earth complexes that are suitable for the purposes of the invention.

The acceptor fluorescent compounds can be chosen from the following group: allophycocyanins, in particular those known under the trade name XL665; luminescent organic molecules, such as rhodamines, cyanines (for instance Cy5), squaraines, coumarins, proflavins, acridines, fluoresceins, boron-dipyrromethene derivatives (sold under the name “Bodipy”), fluorophores known under the name “Atto”, fluorophores known under the name “DY”, compounds known under the name “Alexa”, and nitrobenzoxadiazole. Advantageously, the acceptor fluorescent compounds are chosen from allophycocyanins, rhodamines, cyanines, squaraines, coumarins, proflavins, acridines, fluoresceins, boron-dipyrromethene derivatives, and nitrobenzoxadiazole.

The expressions “cyanines” and “rhodamines” should be respectively understood as “cyanine derivatives” and “rhodamine derivatives”. Those skilled in the art are aware of these various fluorophores, which are commercially available.

The “Alexa” compounds are sold by the company Invitrogen; the “Atto” compounds are sold by the company Atto-tec; the “DY” compounds are sold by the company Dyomics; the “Cy” compounds are sold by the company Amersham Biosciences; the other compounds are sold by various suppliers of chemical reagents, such as the companies Sigma, Aldrich or Acros.

For the purposes of the invention, cyanine derivatives or fluorescein are preferred as acceptor fluorescent compounds.

### EXAMPLE 1

In this example, the method according to the invention was used to quantify the presence of HER2-HER2 dimers in tumor samples from patients, in particular mammary tumors.

The fluorophores Lumi4®Tb and d2 (Cisbio bioassays) were used as respectively donor and acceptor FRET partners. The quantification of the HER2-HER2 homodimers was carried out with a single antibody (trastuzumab, Roche Pharma AG) labeled either with Lumi4®Tb or with d2.

50 μm cryosections of tumor were incubated overnight in 180 μl of TR-FRET buffer (1×PBS/10% BSA+protease inhibitor cocktail, Roche, ref. 1836153) containing 50 nM of each of the two fluorescent conjugates. A DNA stain was then carried out by adding 20 μl of a solution of Hoechst 33342 (Invitrogen) at 0.1 mg/ml and incubating at ambient temperature for 10 min. After washing and centrifugation, the samples were resuspended in the TR-FRET buffer, subjected to sonication and transferred into a microplate.

The Lumi4®Tb and d2 fluorescence signals were measured respectively at 620 and 665 nm in time-resolved mode (delay 60 μs; window 400 μs) after excitation at 337 nm using a Pherastar® fluorimeter (BMG Labtech). The Hoechst 33342 signal was measured in fluorescence mode at 460 nm. These signals were corrected for the background noise according to the formula:

F_{corrected}=Fₛₐₘₚₗₑ−F_{backgroundnoise},

in which the values of F_{backgroundnoise} were obtained by measuring the fluorescence of the TR-FRET buffer alone.

Moreover, for each assay, the fluorescence signals measured on solutions obtained by two-fold cascade dilution (so as to obtain a concentration range) starting from a stock solution containing a mixture of 50 nM of antibody-Lumi4®-Tb and 50 nM of antibody-d2 were measured simultaneously with the samples, and the signal obtained at 665 nm was compared with that measured at 620 nm for each antibody concentration. The resulting curve was used to calculate the contribution of the Lumi4®-Tb fluorescence at 665 nm (F_{665Tb}) on the basis of the signal emitted by the samples at 620 nm. The TR-FRET signal was expressed in the following way:

ΔF₆₆₅=F₆₆₅ₛₐₘₚₗₑ−F_{665Tb}

The signal of the DNA-Hoechst 33342 complex measured at 460 nm (Exc 350 nm/Em 460 nm: F₄₆₀) was used to standardize the TR-FRET signal so as to take into account the variability in the amount of biological material present in each incubation medium and while standardizing to an average value of 100 000 fluorescence units (FU):

TR-FRET_{standardized}=(ΔF₆₆₅×100 000)/(F₄₆₀).

The standardized TR-FRET signal was expressed in FU.

Results: Quantification of the HER2-HER2 Dimers

18 mammary tumor samples were obtained. HER2-HER2 homodimers were detected in 12 (66.7%) of these 18 samples. In most of these samples, the dimer expression levels were low to medium (200 to 2700 FU), except for five samples which showed high levels of HER2-HER2 dimers (range 12 000-32 400 FU). Entirely surprisingly, the HER2-HER2 homodimers were observed not only in the Herceptest™-positive tumor samples, but also in approximately half the Herceptest™-negative tumors (Herceptest™ 0, 1+ or 2+), even though the signals observed were, in those cases, approximately 50 times lower than those measured in the Herceptest™-positive tumor samples.

For each sample, the coefficient of variation of the quantification of the HER2-HER2 dimers was calculated in three independent experiments and an average value of less than 25% for the three tests was obtained.

For the first time, a reliable method for quantifying HER2 dimers makes it possible to quantify the HER2-HER2 dimers, even in Herceptest™-negative patients. It does not have the drawbacks of staining by immunohistochemistry and of the FISH technique and can be carried out in hospital, contrary to the technique of the company Monogram described in patent application WO 2009/086197.

### EXAMPLE 2

The HER2-HER2 dimers were quantified using the method described in example 1 on frozen samples of tumors from 100 patients suffering from breast cancer. Standardized fluorescence signal measurement enabled a quantitative measurement of the HER2-HER2 dimers. The disease-free survival (“DFS”) of the patients and the overall survival (“OS”) were evaluated for each patient.

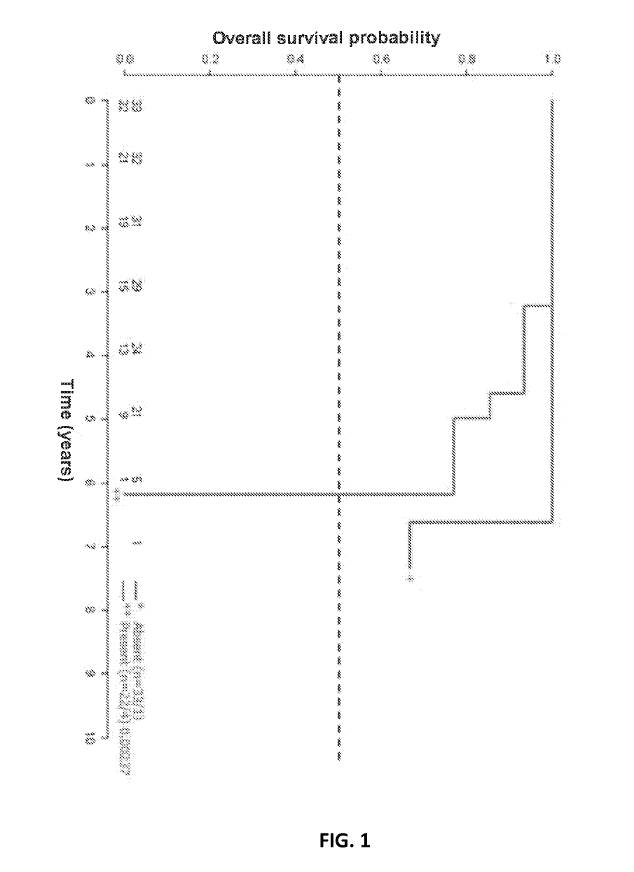

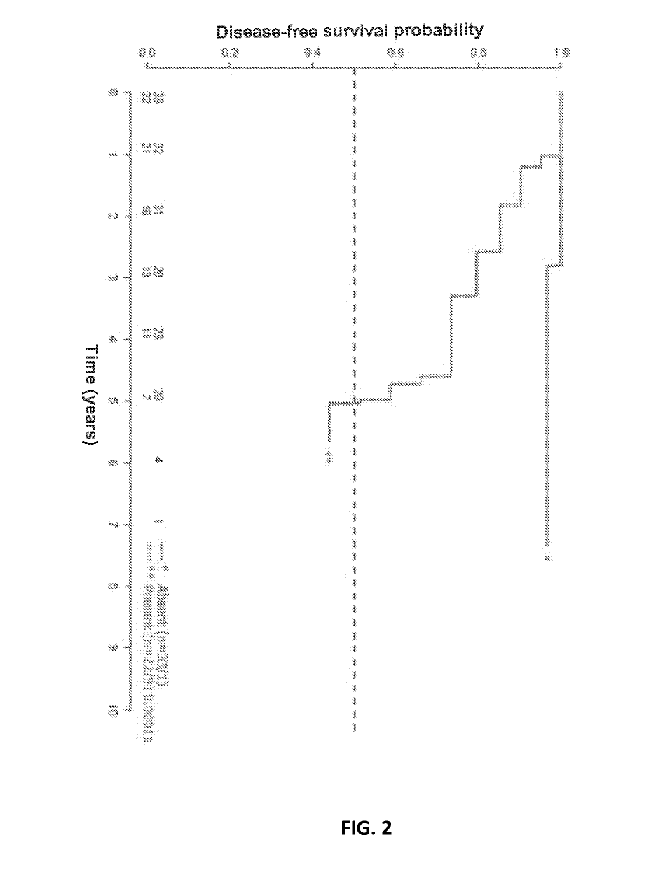

Results: Among the 100 patients, 82 were IHC-HER2-negative (Herceptest™-negative), including 60 patients who were ER-positive and treated with hormone therapy. It was possible to use the samples from 55 of these 60 patients. Using Cox proportional hazards analyses, it was shown that, in the IHC-HER2-negative and ER-positive subjects, the presence of HER2-HER2 dimers was significantly associated both with a reduced overall survival (p=0.00237; cf. FIG. 1) and with a reduced disease-free survival (p=0.00011; cf. FIG. 2).

The quantitative measurement of the expression of HER2 and of HER2-HER2 dimers by means of the method of the invention may make it possible to predict the outcome of the disease in subjects suffering from breast cancers who are IHC-HER2-negative and ER-positive. This biomarker is therefore of use for identifying patients for whom hormone treatment is not sufficiently effective and who might benefit from an adjuvant treatment by anti-HER therapy of Herceptin™ type. One of the major assets of the invention is thus to be able to improve the selection of patients susceptible to being able to respond to this type of treatment.

1. An ex vivo method for determining the susceptibility of a patient suffering from cancer to respond to a therapeutic treatment based on the administration of an antibody specific for the HER2 protein, said method comprising the quantification of HER2-HER2 dimers in a tissue sample from said patient,
wherein said quantification is carried out by the real-time measurement of the fluorescence emitted by a pair of FRET partners brought into contact with said sample, the first member of this pair being directly or indirectly bonded to a first ligand capable of binding to a domain of the HER2 protein, the second member of this pair being directly or indirectly bonded to a second ligand capable of binding to the same domain of the HER2 protein as the first ligand, and in that
wherein the patient is a patient for whom the result of the determination of HER2 expression by immunohistochemistry had proved to be negative.

2. The method of claim 1, wherein the patient has undergone hormone chemotherapy.

3. The method of claim 1, wherein the first ligand and the second ligand are antibodies specific for the same epitope located in the extracellular or intracellular part of HER2.

4. The method of claim 1, wherein the first ligand and the second ligand are identical.

5. The method of claim 1, wherein said first and second ligands are introduced into the incubation medium at a final concentration greater than or equal to 10 nM.

6. The method of claim 1, wherein the quantification of the HER2-HER2 dimers comprises the following steps:
(i) bringing the tissue sample into contact with the pair of FRET partners bonded to the HER2 ligands;
(ii) washing the tissue sample;
(iii) measuring the FRET signal emitted by the measuring medium.

7. The method of claim 6, wherein the quantification of the HER2-HER2 dimers comprises a step of incubating the tissue sample with a labeling agent which emits a signal proportional to the amount of biological material present in the sample, this step being prior to the washing step, and in that the FRET signal is standardized with respect to the signal corresponding to this labeling agent.

8. The method of claim 7, wherein the labeling agent is a fluorescent DNA-labeling agent, and wherein the FRET signal is standardized with respect to the signal corresponding to the fluorescence of this labeling agent.

9. The method of claim 1, which comprises a step of homogenizing the tissue sample in the form of a cell lysate.

10. The method of claim 9, wherein the step of homogenizing the tissue sample is carried out after the introduction of the first and second ligands, and before the measurement of the FRET signal.

11. The method of claim 1, wherein one of the members of the pair of FRET partners is a donor compound which is a rare earth chelate or cryptate.

12. The method of claim 11, wherein the donor compound is a europium or terbium chelate or cryptate.

13. The method of claim 1, wherein one of the members of the pair of FRET partners is an acceptor compound which is selected from the group consisting of allophycocyanins, rhodamines, cyanines, squaraines, coumarins, proflavins, acridines, fluoresceins, boron-dipyrromethene derivatives, fluorophores known under the name “Atto”, fluorophores known under the name “DY”, compounds known under the name “Alexa” and nitrobenzoxadiazole.

14. The method of claim 1, wherein at least one of the FRET partners is covalently bonded to the first ligand or to the second ligand.

15. The method of claim 1, wherein the FRET partners are covalently bonded to the first ligand and to the second ligand.

16. The method of claim 1, wherein the patient is a Herceptest™-negative patient.

17. A kit of reagents which contains a first ligand and a second ligand, each of these ligands being capable of binding specifically to the same domain of the HER2 protein, and these ligands being respectively labeled directly or being suitable for indirect labeling with a donor compound and an acceptor compound, said donor and acceptor compounds forming a pair of FRET partners.

18. The kit of claim 17, wherein at least one of the ligands is covalently labeled with one of the FRET partners.

19. The kit of reagents of claim 18, wherein both ligands are covalently labeled with one of the FRET partners.

20. The kit of reagents of claim 17, wherein the donor compound is a europium or terbium chelate or cryptate.

21. The kit of reagents of claim 17, wherein the acceptor compound is selected from the group consisting of allophycocyanins, rhodamines, cyanines, squaraines, coumarins, proflavins, acridines, fluoresceins, boron-dipyrromethene derivatives, fluorophores known under the name “Atto”, fluorophores known under the name “DY”, compounds known under the name “Alexa” and nitrobenzoxadiazole.
